(19) United States
(12) Patent Application Publication
(10) Pub. No.: US 2025/0154239 A1
(43) Pub. Date: May 15, 2025

(54) METHOD FOR PREVENTING OR TREATING SKIN AGING OR INFLAMMATION OR INCREASING A CONTENT OF COLLAGEN

(71) Applicants: National Yang Ming Chiao Tung University, Hsinchu County (TW); Taipei Veterans General Hospital, Taipei City (TW)

(72) Inventors: Ting-Ting CHANG, Hsinchu County (TW); Jaw-Wen CHEN, Hsinchu County (TW)

(73) Assignees: National Yang Ming Chiao Tung University, Hsinchu County (TW); Taipei Veterans General Hospital, Taipei City (TW)

(21) Appl. No.: 18/805,442

(22) Filed: Aug. 14, 2024

(30) Foreign Application Priority Data
Nov. 9, 2023 (TW) 112143277
Jul. 19, 2024 (TW) 113127202

(51) Int. Cl. C07K 16/24; A61K 8/64; A61K 39/00; A61P 17/02; A61P 29/00; A61Q 19/08

(52) U.S. Cl. CPC C07K 16/24; A61K 8/64; A61P 17/02; A61P 29/00; A61Q 19/08; A61K 2039/505; C07K 2317/76

(57) ABSTRACT

Provided is a method for treating or preventing skin aging or skin inflammation in an individual in need thereof, which includes administering an effective amount of a pharmaceutical composition to the individual. The pharmaceutical composition includes a CCL4 antagonist and/or a pharmaceutically acceptable salt thereof and a pharmaceutically acceptable carrier thereof. Also provided is a method for increasing a content of collagen in an individual in need thereof, which includes administering an effective amount of a cosmetic composition to the individual.

### CROSS-REFERENCE TO RELATED APPLICATIONS

This application claims the benefit of priority to Taiwan Patent Application No. 112143277, filed Nov. 9, 2023, and Taiwan Patent Application No. 113127202, filed Jul. 19, 2024, the entire contents of which are hereby incorporated by reference.

### INCORPORATION BY REFERENCE OF A SEQUENCE LISTING XML

The present disclosure is being filed along with a Sequence Listing in electronic format. The Sequence Listing is provided as a file entitled 1013357_0120_SL_14AUG2024, created on Aug. 14, 2024, and is 3.57 KB in size. The contents in the electronic format of the Sequence Listing are incorporated by reference herein in their entirety.

### BACKGROUND

### 1. Technical Field

The present disclosure relates to prevention, improvement and/or treatment of skin aging or inflammation; in particular, the disclosure relates to a method and use of preventing, improving and/or treating skin aging or inflammation by increasing collagen content in the skin by administering a therapeutically effective amount of a chemokine (C-C motif) ligand 4 (CCL4) antagonist.

### 2. Description of Related Art

As people age, their skin ages gradually. Besides affecting the appearance of skin, skin aging is also accompanied by unfavorable conditions such as poor blood circulation, increasing inflammation, loss of collagen, and slow wound healing.

Due to the advancement of modern medical technology and improvement of living standards, the life span of human beings has extended substantially. The elderly population is already about 10% of the 8 billion current global population. According to projections from the United Nations, the proportion of the elderly population will increase to about 16% of the global population in 2050. By then, nearly 1.6 billion people will be bothered by skin appearance changes such as collagen loss, rough skin, and skin laxity as they age. Disadvantageously, their daily lives can be negatively affected by unfavorable conditions such as skin inflammation and slow wound healing.

To slow down and treat skin aging, many kinds of topical cosmetic products applied to skin surfaces have been developed in the industry. However, the molecular structures of these cosmetic products are usually too large to be absorbed by skin, and thus cannot meet the expectations of consumers. In addition, many people improve their skin condition by injecting substances such as botulinum toxin, collagen, hyaluronic acid, etc. However, these invasive methods are prone to side effects such as skin trauma and postoperative complications, and their disadvantages include long recovery periods and short-term effects.

Chemokines are a family of cytokines, which are small molecule proteins secreted by cells. They induce chemotaxis of immune cells and can mediate their migration and positioning. Chemokines can be divided into several subfamilies, wherein CCL4 (C-C chemokine ligand 4) belongs to the CC subfamily. CCL4 is a macrophage inflammatory protein, which is known to play a key role in inflammation and immunomodulation. However, there is currently no definite conclusion on the role of chemokines such as CCL4 in skin aging.

Therefore, there is still an urgent need in the industry for effective methods to prevent and treat skin aging and inflammation and to increase content of collagen, so as to improve skin condition and promote wound healing.

### SUMMARY

The present disclosure provides a method for treating or preventing skin aging or skin inflammation in an individual in need thereof, which includes administering an effective amount of a pharmaceutical composition to the individual, and the pharmaceutical composition includes a CCL4 antagonist and/or a pharmaceutically acceptable salt thereof, and a pharmaceutically acceptable carrier thereof.

In addition, the present disclosure provides a use of a pharmaceutical composition in the manufacture of a medicament for treating or preventing skin aging or skin inflammation, and the pharmaceutical composition includes a CCL4 antagonist and/or a pharmaceutically acceptable salt thereof, and a pharmaceutically acceptable carrier thereof.

In at least one embodiment of the present disclosure, the CCL4 antagonist is at least one selected from the group consisting of a CCL4 neutralizing antibody, a CCL4 RNA interference (RNAi), a CCL4 small molecule antagonist, a CCR (C-C motif chemokine receptor) 1 antagonist, a CCR2 antagonist and a CCR5 antagonist.

In at least one embodiment of the present disclosure, the skin aging includes skin laxity, dry skin, rough skin, or slow wound healing.

In at least one embodiment of the present disclosure, administration of the pharmaceutical composition or the medicament promotes angiogenesis, promotes wound healing, increases a content of collagen, reduces expression of an inflammatory factor, and/or reduces expression of an aging factor.

In at least one embodiment of the present disclosure, the pharmaceutical composition or the medicament is in a form for oral administration, intravenous injection, hypodermic injection, intradermic injection, intramuscular injection, intraperitoneal injection, or transdermal administration.

In at least one embodiment of the present disclosure, the CCL4 antagonist and/or a pharmaceutically acceptable salt thereof is present in the pharmaceutical composition in an amount of 0.01 to 80 wt % based on the total weight of the pharmaceutical composition. In another embodiment, the CCL4 antagonist and/or a pharmaceutically acceptable salt thereof is present in the pharmaceutical composition in an amount of 0.1 to 60 wt % based on the total weight of the pharmaceutical composition.

In at least one embodiment of the present disclosure, the CCL4 antagonist and/or a pharmaceutically acceptable salt thereof is administrated in an amount of 0.01 mg/kg to 100 mg/kg.

In at least one embodiment of the present disclosure, the pharmaceutical composition or the medicament is administered 1 to 25 times per week. In at least one embodiment of the present disclosure, the pharmaceutical composition or the medicament is continuously administered for a period of 2 to 4 weeks.

The present disclosure also provides a method for increasing a content of collagen in an individual in need thereof, which includes administering an effective amount of a cosmetic composition to the individual, and the cosmetic composition includes a CCL4 antagonist and/or a cosmetically acceptable salt thereof and a cosmetically acceptable carrier thereof.

The present disclosure also provides a use of a CCL4 antagonist and/or a cosmetically acceptable salt thereof in the manufacture of a cosmetic composition for increasing content of collagen, and the cosmetic composition includes a CCL4 antagonist and/or a pharmaceutically acceptable salt thereof, and a pharmaceutically acceptable carrier thereof.

In at least one embodiment of the cosmetic composition of the present disclosure, the CCL4 antagonist is at least one selected from the group consisting of a CCL4 neutralizing antibody, a CCL4 RNAi agent, a CCL4 small molecule antagonist, a CCR1 antagonist, a CCR2 antagonist and a CCR5 antagonist.

In at least one embodiment of the cosmetic composition of the present disclosure, the CCL4 antagonist and/or a cosmetically acceptable salt thereof is present in an amount of 0.01 to 15 wt % based on the total weight of the cosmetic composition.

In at least one embodiment of the cosmetic composition of the present disclosure, the CCL4 antagonist and/or a cosmetically acceptable salt thereof is administrated in an amount of 0.01 mg/kg to 100 mg/kg.

In at least one embodiment of the cosmetic composition of the present disclosure, the cosmetic composition is administered 1 to 25 times per week. In at least one embodiment of the present disclosure, the cosmetic composition is continuously administered for a period of 1 to 8 weeks.

According to the present disclosure, inhibition of the CCL4 is effective for promoting angiogenesis, promoting wound healing, increasing the content of collagen, reducing the expression of inflammatory factors, and/or reducing the expression of aging factors. Therefore, administering any CCL4 antagonist (any antibody, ribonucleic acid, small molecule protein, compound, etc. having an ability of inhibiting the activity of CCL4) to an individual can achieve the effect of inhibiting CCL4, thereby treating or preventing skin aging or skin inflammation and increasing the content of collagen.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present disclosure can be more fully understood by reading the following description of the embodiments, with reference made to the accompanying drawings.

FIG. 1 shows the profile of the concentration of CCL4 in serum of WT6M group (6-month-old young C57BL/6JNarl mice with wild-type gene), CCL4KO6M group (6-month-old young C57BL/6JNarl mice with CCL4 gene knocked out), WT18M group (18-month-old aging C57BL/6JNarl mice with wild-type gene) and CCL4KO18M group (18-month-old aging C57BL/6JNarl mice with CCL4 gene knocked out). The results show that the CCL4 concentration in serum of WT18M group is higher than that of WT6M group, and CCL4 is almost undetectable in the serum of CCL4KO6M group and CCL4KO18M group. n=6 for each group. ** represents p<0.01.

FIGS. 2A and 2B show conditions of wound healing in WT6M group, CCL4KO6M group, WT18M group, and CCL4KO18M group. FIG. 2A shows photographs of the wounds of mice in each group on days 0, 1, 3, and 7, respectively.

FIG. 2B is a bar chart indicating the percentage of healed wound area of mice in each group on day 7, which illustrates that inhibiting CCL4 can promote wound healing in aging mice. n=6 for each group. * represents p<0.05.

FIG. 3 shows biopsies of representative wound area of WT6M group, CCL4KO6M group, WT18M group, and CCL4KO18M group. These biopsies were stained with H&E. Locations and sizes of wound are marked with arrows, which illustrate that inhibiting CCL4 can promote wound healing in aging mice.

FIG. 4 shows biopsies of representative wound area of WT6M group, CCL4KO6M group, WT18M group, and CCL4KO18M group. These biopsies were immunostained with anti-CD31 antibodies, of which CD31 is a biological indicator of microvessels, which illustrates that inhibiting CCL4 can promote angiogenesis in aging mice.

FIG. 5 shows biopsies of representative wound area of WT6M group, CCL4KO6M group, WT18M group, and CCL4KO18M group. These biopsies were immunostained with anti-Ki67 antibodies, of which Ki67 is a biological indicator of cell proliferation, which illustrates that inhibiting CCL4 can promote cell proliferation in aging mice.

FIG. 6 shows biopsies of representative wound area of WT6M group, CCL4KO6M group, WT18M group, and CCL4KO18M group. These biopsies were stained by Masson's trichrome staining method. Collagen is shown in blue, which illustrates that inhibiting CCL4 can increase the contents of collagen in aging mice.

FIG. 7 is an image of western blotting and quantification thereof showing contents of angiogenesis factor (p-AKT (relative to AKT), VEGF) association proteins in the wound area of WT6M group, CCL4KO6M group, WT18M group, and CCL4KO18M group, which illustrates that inhibiting CCL4 can increase the contents of angiogenesis factor association proteins in aging mice, thereby promoting angiogenesis. n=3 for each group. * represents p<0.05.

FIG. 8 is an image of western blotting and quantification thereof showing contents of aging factor (SIRT1, p53) association proteins in the wound area of WT6M group, CCL4KO6M group, WT18M group, and CCL4KO18M group, which illustrates that inhibiting CCL4 can increase the contents of aging factor association proteins and reduce the contents of aging factor association proteins in aging mice, thereby improving skin aging. n=3 for each group. * represents p<0.05. ** represents p<0.01.

FIG. 9 is an image of western blotting and quantification thereof showing contents of inflammatory factor (IL-6, TNF-α) association proteins in the wound area of WT6M group, CCL4KO6M group, WT18M group, and CCL4KO18M group, which illustrates that inhibiting CCL4 can reduce the contents of inflammatory factor association proteins, thereby improving skin inflammation. n=3 for each group. * represents p<0.05. ** represents p<0.01.

FIGS. 10A and 10B show conditions of wound healing in WT6M group, WT6M+CCL4 mAb group, WT18M group, and WT18M+CCL4 mAb group. FIG. 10A illustrates photographs of the wounds of mice in each group on days 0, 1, 3, and 7, respectively. FIG. 10B is a bar chart indicating the percentage of healed wound area of mice in each group on day 7, which illustrates that administering CCL4 antagonist can promote wound healing in aging mice. n=6 for each group. ** represents p<0.01.

FIG. 11 shows biopsies of representative wound area of WT6M group, WT6M+CCL4 mAb group, WT18M group, and WT18M+CCL4 mAb group. These biopsies were stained with H&E. Locations and sizes of wound are marked with arrows, which illustrate that administering CCL4 antagonist can promote wound healing in aging mice.

FIG. 12 shows biopsies of representative wound area of WT6M group, WT6M+CCL4 mAb group, WT18M group, and WT18M+CCL4 mAb group. These biopsies were immunostained with anti-CD31 antibodies (CD31 is a biological indicator of microvessels), which illustrates that administering CCL4 antagonist can promote angiogenesis in aging mice.

FIG. 13 shows biopsies of representative wound area of WT6M group, WT6M+CCL4 mAb group, WT18M group, and WT18M+CCL4 mAb group. These biopsies were immunostained with anti-Ki67 antibodies (Ki67 is a biological indicator of cell proliferation), which illustrates that administering CCL4 antagonist can promote cell proliferation in aging mice.

FIG. 14 shows biopsies of representative wound area of WT6M group, WT6M+CCL4 mAb group, WT18M group, and WT18M+CCL4 mAb group. These biopsies were stained by Masson's trichrome staining method. Collagen is shown in blue, which illustrates that administering CCL4 antagonist can increase the contents of collagen in aging mice.

FIG. 15 is an image of western blotting and quantification thereof showing contents of angiogenesis factor (p-AKT (relative to AKT), VEGF) association proteins in the wound area of WT6M group, WT6M+CCL4 mAb group, WT18M group, and WT18M+CCL4 mAb group, which illustrates that administering CCL4 antagonist can increase the contents of angiogenesis factor association proteins in aging mice, thereby promoting angiogenesis. n=3 for each group. * represents p<0.05. ** represents p<0.01.

FIG. 16 is an image of western blotting and quantification thereof showing contents of aging factor (SIRT1, p53) association proteins in the wound area of WT6M group, WT6M+CCL4 mAb group, WT18M group, and WT18M+CCL4 mAb group, which illustrates that administering CCL4 antagonist can increase the contents of aging factor association proteins and reduce the contents of aging factor association proteins in aging mice, thereby improving skin aging. n=3 for each group. * represents p<0.05. ** represents p<0.01.

FIG. 17 is an image of western blotting and quantification thereof showing contents of inflammatory factor (IL-6, TNF-α) association proteins in the wound area of WT6M group, WT6M+CCL4 mAb group, WT18M group, and WT18M+CCL4 mAb group, which illustrates that administering CCL4 antagonist can reduce the contents of inflammatory factor association proteins, thereby improving skin inflammation. n=3 for each group. ** represents p<0.01.

### DETAILED DESCRIPTION

The following examples are used to illustrate the present disclosure. A person skilled in the art can easily conceive of the other advantages and effects of the present disclosure based on the specification, claims, and drawings. The present disclosure can also be implemented or applied as described in various examples below. One of skill in the art will appreciate that it is possible to modify or alter the following examples for carrying out the present disclosure without violating its spirit and scope, for different aspects and applications.

All terms used herein, including descriptive or technical terms, shall be construed as having meanings that are evident to a person with ordinary skill in the art. However, these terms can have different meanings according to the intention of a person of ordinary skill in the art, case precedents, or the appearance of new technology. In addition, some terms can be arbitrarily selected by applicant. In this case, the meaning of the selected terms will be described in detail in the descriptions of the present disclosure. Thus, the terms used herein have to be defined based on the meaning thereof together with the descriptions throughout the specification.

As used in the present disclosure, the singular forms “a,” “an” and “the” include plural referents unless expressly and unequivocally limited to one referent. The term “or” is used interchangeably with the term “and/or” unless the context clearly indicates otherwise.

As used herein, the term “include” or “comprise” are used in reference to compositions, methods, and respective component(s) thereof, which are open to the inclusion of additional unspecified elements, whether essential or not.

The term “individual” is used interchangeably with the term “patient” in the present disclosure, and the term “individual” refers to a human being or an animal. Examples of individuals include but not limited to: humans, monkeys, mice, rats, marmots, ferrets, rabbits, hamsters, cows, horses, pigs, deer, dogs, cats, foxes, wolves, chickens, emus, ostriches and fish. In certain embodiments of the present disclosure, the individual is a mammal, such as, for example, a primate or a human.

The term “treat” includes alleviation or elimination of illness, disease or condition, or one or more symptoms associated thereof; or alleviation or eradication of the cause thereof.

The term “prevent” includes methods of postponing and/or preventing the attack of illness, disease or condition and/or attendant symptoms thereof, preventing an individual from suffering therefrom, or reducing an individual's risk of suffering therefrom.

The terms “therapeutically effective amount” or “effective amount” refer to the amount of an active ingredient (e.g., a CCL4 antagonist) sufficient to prevent or treat the progress of illness, disease or condition or to alleviate the illness, disease or condition to a certain extent when administered to an individual. As is known to those with ordinary skill in the art, the effective amount can vary depending on the route of administration, the excipients used, other components co-administered, and the condition being treated.

The numerical ranges used herein are inclusive and combinable, and any numerical value that falls within the numerical range herein can be used as a maximum or minimum value to derive subrange therefrom. For instance, the numerical range “0.01 mg to 1000 mg” should be understood to include any subrange between the minimum value 0.01 mg and the maximum value 1000 mg, such as from 0.01 mg to 800 mg, from 0.5 mg to about 700 mg, from 1 mg to about 600 mg. In addition, various numerical values herein can be selected optionally as the highest and lowest values of a derived numerical range. For instance, the values 0.01 mg, 5 mg, and 30 mg can be derived to numerical ranges 0.01 mg to 5 mg, 0.01 mg to 30 mg, and 5 mg to 30 mg.

The terms “about” or “approximately” refer to the acceptable error for a particular value determined by those with ordinary skill in the art, depending partly on how the value is measured or determined. In some embodiments, the terms “about” or “approximately” refer to range of 20%, 15%, 10%, 9%, 8%, 7%, 6%, 5%, 4%, 3%, 2%, 1%, 0.5% or 0.05% of a given value.

The method of the present disclosure treats or prevents skin aging or skin inflammation, and/or increasing a content of collagen in an individual in need thereof by a CCL4 antagonist and/or a pharmaceutically acceptable salt thereof. The CCL4 antagonist refers to any antibody, ribonucleic acid, small molecule protein, compound, etc. that can inhibit the activity of CCL4, for example but not limited to a CCL4 neutralizing antibody, a CCL4 RNAi agent, a CCL4 small molecule antagonist, a CCR1 antagonist, a CCR2 antagonist, and a CCR5 antagonist. In one embodiment of the present disclosure, the CCL4 antagonist can inhibit the binding of CCL4 to a receptor thereof. In another embodiment of the present disclosure, the CCL4 antagonist can inhibit production of intracellular signaling after CCL4 binds to the receptor thereof. For instance, the CCL4 antagonist can act on at least one of CCL4 and a receptor of CCL4, thereby blocking the signaling of CCL4. As used herein, the CCL4 receptor includes but not limited to CCR1, CCR2, and CCR5.

As used herein, “CCR1” or “CCR1 receptor,” “CCR2” or “CCR2 receptor” and “CCR5” or “CCR5 receptor” are used interchangeably and have general meanings as known in the art. CCR1, CCR2 and CCR5 receptors can be derived from any sources but are typically from mammalian (such as humans or non-human primates). In some embodiments of the present disclosure, CCR1, CCR2 and CCR5 receptors are human receptors.

As used herein, the term “CCL4” has general meaning in the art. CCL4 is a ligand of CCR1, CCR2 and CCR5 receptors and can come from any sources but is typically CCL4 of mammalian (such as human or non-human primate). In one embodiment of the present disclosure, the CCL4 is human CCL4.

As used herein, the term “skin aging” refers to conditions of skin laxity, dry skin, rough skin, or slow wound healing, which may be a natural aging phenomenon due to age and exposure to sun, or can be caused by disease or drug action. When an individual or patient experiences “skin aging,” their skin can also suffer from poor blood circulation, increased inflammation, loss of collagen, and other unfavorable conditions.

As used herein, the term “skin inflammation” refers to a inflammation condition of the skin, including acute inflammation caused by injury, infection, etc., and chronic inflammation caused by aging, stress, daily routine, etc.

In at least one embodiment, the present disclosure provides a method for treating or preventing skin aging or skin inflammation in an individual in need thereof, including administering an effective amount of a pharmaceutical composition to the individual. The pharmaceutical composition includes a CCL4 antagonist and/or a pharmaceutically acceptable salt thereof, along with a pharmaceutically acceptable carrier thereof. The method can promote angiogenesis, promote wound healing, increase a content of collagen, reduce expression of inflammatory factors and/or reduce expression of aging factors by administering the CCL4 antagonist and/or a pharmaceutically acceptable salt thereof, thereby achieving the effect of treating or preventing skin aging or skin inflammation in the individual. In some embodiments, the CCL4 antagonist and/or a pharmaceutically acceptable salt thereof is administered topically to the individual. In some embodiments, the CCL4 antagonist and/or a pharmaceutically acceptable salt thereof is topically applied to the skin of the individual. In some embodiments, the CCL4 antagonist and/or a pharmaceutically acceptable salt thereof is topically applied to a selected area of the individual's skin to treat or prevent skin aging or skin inflammation at a target part of the individual's body.

Collagen is an important protein in human body, which is used for maintaining body functions, facilitating growth of bone, maintaining a density of bone, maintaining tendons, and preventing joint wear. It also has the effects of keeping the skin moist, maintaining elasticity, supporting skin, helping reduce wrinkles, and firming skin. When collagen is lost, skin would become lax, dry, rough, and wrinkled. General methods of collagen supplementation in the market are oral administration, application and injection of collagen. However, oral administration and application of collagen are difficult for individuals to absorb collagen into the skin, and there is still a large risk of side effects for injection method. Besides, injection must be operated by professionals, and thus accompanying the disadvantages of inconvenience and high price.

In at least one embodiment, the present disclosure provides a method for increasing a content of collagen in an individual in need thereof, which includes administering an effective amount of a cosmetic composition to the individual. The cosmetic composition includes a CCL4 antagonist and/or a cosmetically acceptable salt thereof, and a cosmetically acceptable carrier thereof. This method achieves the effect of increasing the content of collagen directly in the individual by administering the CCL4 antagonists and/or a cosmetically acceptable salt thereof. In some embodiments, the CCL4 antagonist and/or a cosmetically acceptable salt thereof is administered topically to the individual. In some embodiments, the CCL4 antagonist and/or a cosmetically acceptable salt thereof is topically applied to the skin of the individual. In some embodiments, the CCL4 antagonist and/or a cosmetically acceptable salt thereof is topically applied to a selected area of the individual's skin to increase the content of collagen at a target part of the individual's body.

In some embodiments of the present disclosure, the methods provided by the present disclosure can be used in individuals of various ages, preferably for elder individuals.

In some embodiments of the present disclosure, the CCL4 antagonist and/or a salt thereof is present in the cosmetic composition or pharmaceutical composition in an amount of about 0.01 to about 80 wt % based on a total weight of the cosmetic composition or pharmaceutical composition, such as about 0.01 to about 60 wt %, about 0.01 to about 30 wt %, about 0.01 to about 15 wt %, about 0.1 wt % to about 60 wt %, about 0.1 wt % to about 40 wt %, about 0.1 wt % to about 20 wt %, about 0.5 wt % to about 70 wt %, about 0.5 wt % to about 50 wt %, about 0.5 wt % to about 30 wt %, about 1 wt % to about 75 wt %, about 1 wt % to about 60 wt %, about 1 wt % to about 45 wt %, about 5 wt % to about 50 wt %, about 10 wt % to about 40 wt %, about 15 wt % to about 30 wt %. In another embodiment, the lower limit of the effective amount per kg weight of the CCL4 antagonist and/or a salt thereof administrated to the individual is selected from 0.01 mg/kg, 0.05 mg/kg, 0.1 mg/kg, 0.5 mg/kg, 1 mg/kg, 2 mg/kg, 2.5 mg/kg, 5 mg/kg, 10 mg/kg, 15 mg/kg, 20 mg/kg and 25 mg/kg, and the upper limit is selected from 1000 mg/kg, 900 mg/kg, 800 mg/kg, 700 mg/kg, 600 mg/kg, 500 mg/kg, 400 mg/kg, 300 mg/kg, 200 mg/kg, 100 mg/kg, 90 mg/kg, 80 mg/kg, 70 mg/kg, 60 mg/kg, 50 mg/kg, 40 mg/kg and 30 mg/kg. Preferably, the CCL4 antagonist and/or a salt thereof is administered in an amount of 0.01 mg/kg to 100 mg/kg; more preferably, the CCL4 antagonist and/or a salt thereof is administered in an amount of 2 mg/kg to 10 mg/kg; even more preferably, the CCL4 antagonist and/or a salt thereof is administered in an amount of 2.5 mg/kg to 5 mg/kg.

In one embodiment of the present disclosure, the CCL4 antagonist and/or a pharmaceutically or cosmetically acceptable salt thereof is administered 1 to 4 times per day, such as once per day, twice per day, three times per day or four times per day. In another embodiment, the CCL4 antagonist and/or a pharmaceutically or cosmetically acceptable salt thereof is administered at 1 to 25 times per week, for example, once per week, twice per week, 3 times per week, 4 times per week, 5 times per week, 6 times per week, 7 times per week, 8 times per week, 9 times per week, 10 times per week, 11 times per week, 12 times per week, 13 times per week, 14 times per week, 15 times per week, 16 times per week, 17 times per week, 18 times per week, 19 times per week, 20 times per week, 21 times per week, 22 times per week, 23 times per week, 24 times per week, or 25 times per week. For instance, the CCL4 antagonist and/or a pharmaceutically or cosmetically acceptable salt thereof is administered two times a day or five times a week. In one embodiment of the present disclosure, the pharmaceutical composition, medicament, or cosmetic composition including the CCL4 antagonist and/or a pharmaceutically or cosmetically acceptable salt thereof is administered 1 to 5 times per week, or administered 2 to 7 times per week.

In at least one embodiment of the present disclosure, the pharmaceutical composition or medicament including the CCL4 antagonist and/or a pharmaceutically acceptable salt thereof is administered continuously for 2 to 4 weeks to treat or prevent skin aging or skin inflammation. In at least one embodiment of the present disclosure, the cosmetic composition including the CCL4 antagonist and/or a cosmetically acceptable salt thereof is administered continuously for 1 to 8 weeks to increase a content of collagen.

As used herein, the term “administration” refers to administering an active ingredient (such as a CCL4 antagonist and/or a salt thereof) to an individual by a method or route that makes the active ingredient located at least partially in a desired location to produce a desired effect. The active ingredients provided by the present disclosure can be administered by any suitable route known in the art, including but not limited to oral, intravenous injection, hypodermic injection, intradermic injection, intramuscular injection, intraperitoneal injection, or transdermal administration.

The cosmetic composition or pharmaceutical composition provided by the present disclosure can be formulated into any dosage form suitable for topical administration for topical or systemic effects, including tablets, pills, granules, sublingual tablets, film coated tablets, injections, emulsions, solutions, suspensions, creams, gels, hydrogels, ointments, powders, dressings, elixirs, lotions, tinctures, pastes, foams, films, aerogels, irrigations, sprays, suppositories, bandages and skin patches.

Cosmetic products or pharmaceutically acceptable carriers suitable for the topical formulations provided by the present disclosure include but not limited to aqueous carriers, water-miscible carriers, non-aqueous carriers, antimicrobial agents, preservatives, stabilizers, solubility enhancers, isotonic agents, buffers, antioxidants, topical anesthetics, suspending agents, dispersants, wetting agents, emulsifiers, complexing agents, chelating agents, penetration enhancers, cryoprotectants, lyoprotectants, thickeners and inert gases.

Cosmetic compositions or pharmaceutical compositions can also be administered topically by electroporation, iontophoresis, sonophoresis, phonophoresis, microneedle or needle-free injection, such as PowderTect (Chiron Corp., Emeryville, CA) and Bioject (Bioject Medical Technologies Inc., Tualatin, OR).

The cosmetic composition or pharmaceutical composition provided by the present disclosure can be provided in the form of ointments, creams and gels. Suitable ointment excipients include oily or hydrocarbon-containing excipients, including lard, benzoated lard, olive oil, cottonseed oil and other oils, white petrolatum; emulsifiable or absorbable excipients, such as hydrophilic petrolatum, hydroxystearin sulfate, and anhydrous lanolin; water-removable excipients, such as hydrophilic ointments; water soluble ointment excipients, including polyethylene glycols of different molecular weights; emulsion excipients, water-in-oil (W/O) emulsions or oil-in-water (O/W) emulsions, including cetyl alcohol, glyceryl monostearate, lanolin, and stearic acid (see Remington: The Science and Practice of Pharmacy). These excipients are emollients, but often require addition of antioxidants and preservatives.

Suitable cream base may be oil in water or water in oil. Suitable cream excipient may be water washable, and contains an oil phase, an emulsifier and an aqueous phase. The oil phase is also called the “internal” phase, which usually comprises of petrolatum and a fatty alcohol such as cetyl alcohol or stearyl alcohol. Although not essential, the aqueous phase usually exceeds the oil phase in volume and often contains a wetting agent. Emulsifier in cream formulation may be nonionic, anionic, cationic or amphoteric surfactant.

Gel is a semi-solid suspension system. Single-phase gel contains organic macromolecules substantially distributed uniformly throughout the liquid excipient. Suitable gelling agents include but are not limited to cross-linked acrylic polymers such as Carbomer, carboxypolyalkylene and Carbopol; hydrophilic polymers such as polyethylene oxide, polyoxyethylene-polyoxypropylene copolymer and polyvinyl alcohol; cellulosic polymers such as hydroxypropyl cellulose, hydroxyethyl cellulose, hydroxypropyl methyl cellulose, hydroxypropyl methyl cellulose phthalate, and methyl cellulose; gums, such as tragaca nth gum and xanthan gum; sodium alginate; and gelatin. In order to prepare a uniform gel, a dispersant such as alcohol or glycerin can be added, or the gelling agent can be dispersed by grinding, mechanical mixing and/or stirring.

Forms of cosmetic composition or pharmaceutical composition suitable for injection use include sterile aqueous solutions or dispersions, and sterile powders used for instant preparation of sterile injection solutions or dispersions. In all cases, the form must be sterile and easily injectable fluid. It must remain stable under preparation and storage conditions, and must be protected from contamination by microorganisms such as bacteria and fungi. The carrier may be solvent or dispersion medium, which contains water, ethanol, polyols (such as glycerin, propylene glycol, liquid polyethylene glycol, etc.), and suitable mixtures thereof, and vegetable oils. Various antibacterial and antifungal agents such as paraben, chloretone, phenol, sorbic acid, thimerosal, etc. can be used if required. In many cases, it is preferred to include isotonic agents such as sugar or sodium chloride. The absorption of injection compositions can be prolonged by using agents such as aluminum monostearate and gelatin that prolonged absorption in the compositions.

Many examples have been used to illustrate the present disclosure. The examples below should not be taken as a limit to the scope of the present disclosure.

### Examples

### Preparation of Animal Models

Six-week-old wild-type C57BL/6JNarl mice and CCL4KO mice (C57BL/6JNarl-Ccl4em1 gene knocked out mice) were designed and purchased from the National Laboratory Animal Center (Taipei, Taiwan). The CCL4KO mouse is a mouse with a C57BL/6JNarl genetic background, and the CCL4 gene was knocked out by using the CRISPR/Cas9 system. All mice were sequenced by PCR using specific primers (forward primer: 5′-TCTCCCTCCTTTCTCTTCCGTG-3′, and reverse primer: 5′-TCTACTCCCATGATGGCTGACC-3′).

### (1) Grouping Animals for Gene Knock Out Experimentation

Twelve C57BL/6JNarl mice and twelve CCL4KO mice were each divided into two groups (a total of four groups, 6 mice in each group), and one group from each of the C57BL/6JNarl mice and CCL4KO mice was selected as the young group, and the remaining mice were the aging group. The experimentation began when the young group and aging group were six months old and eighteen months old, respectively. The groups are shown in Table 1 below.

TABLE 1

| | | Group names shown |
|---|---|---|
| | | in the following |
| | | embodiments and |
| | Genotype | drawings |
| Young group | Wild-type C57BL/6JNarl mice | WT6M group |
| (Six-month-old) | CCL4KO mice | CCL4KO6M group |
| Aging group | Wild-type C57BL/6JNarl mice | WT18M group |
| (Eighteen- | CCL4KO mice | CCL4KO18M group |
| month-old) | | |

### (2) Grouping Animals for Neutralizing Antibody Experimentation

Twenty-four C57BL/6JNarl mice with an average weight of 30 g were divided into four groups, with 6 mice in each group, in which two groups were selected as the young groups, and the remaining mice were the aging groups. The experimentation began when the young group and aging group were six months old and eighteen months old, respectively, in which one group from each of the young groups and the aging groups were intraperitoneally injected with 100 g of CCL4 monoclonal neutralizing antibody (R&D Systems, MAB451, Minneapolis, MN, USA) three times per week for 2 weeks. The groups are shown in Table 2 below.

TABLE 2

| | | Injected with CCL4 | Group names shown in the |
|---|---|---|---|
| | | monoclonal | following embodiments |
| | Genotype | neutralizing antibody | and drawings |
| Young group | Wild-type | No | WT6M group |
| (Six-month- | C57BL/6JNarl mice | | |
| old) | Wild-type | Yes | WT6M + CCL4 mAb group |
| | C57BL/6JNarl mice | | |
| Aging group | Wild-type | No | WT18M group |
| (Eighteen- | C57BL/6JNarl mice | | |
| month-old) | Wild-type | Yes | WT18M + CCL4 mAb |
| | C57BL/6JNarl mice | | group |

According to standards of the Institutional Animal Care & Use Committee of National Yang Ming Chiao Tung University, all animals were raised in the Laboratory Animal Center of National Yang Ming Chiao Tung University in a sterile environment, living in microisolator cages, and 12 hours was set as diurnal cycle. The following animal experiments were approved by the Institutional Animal Care & Use Committee of National Yang Ming Chiao Tung University (IACUC No. 1101114).

### Statistical Analysis

Result data in the following embodiments were shown as mean value±standard deviation. Statistical analysis was performed by unpaired Student's t test or analysis of variance, followed by Scheffe's multiple comparison post hoc test. Data were analyzed using SPSS software (version 14). Statistical significance was considered when the p value was <0.05.

### Example 1: Determination of CCL4 Concentration in Serum

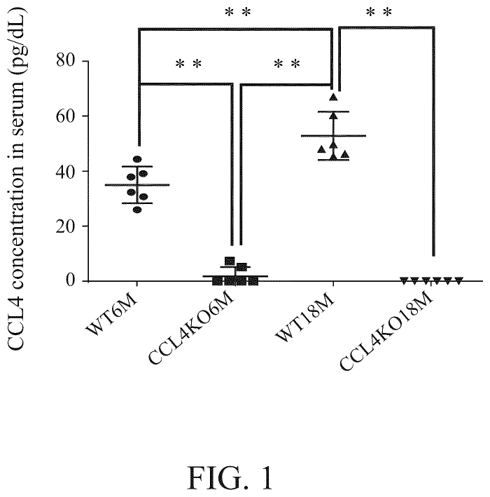

Blood was collected from the mice in WT6M group, CCL4KO6M group, WT18M group, and CCL4KO18M group established in the above example “Preparation of animal models (1),” and serum was separated from the collected blood. Concentrations of CCL4 in the serum of mice in each group were determined by the CCL4 ELISA kit (R&D, Minneapolis, MN, USA) following the manufacturer's ELISA test instructions. The results are shown in FIG. 1 (n=6, ** represents p<0.01).

As shown in FIG. 1, in wild-type mice, the CCL4 concentration in serum of the aging group (WT18M group) was significantly higher comparing to the young group (WT6M group). However, in CCL4KO mice, CCL4 was almost undetectable in either the young group (CCL4KO6M group) or the aging group (CCL4KO18M group). The foregoing results showed that the concentration of CCL4 in animals increased with age. In the present disclosure, CCL4 concentration in the body of mouse model prepared by knocking out the CCL4 gene can be reduced successfully, and the CCL4 concentration would not change with increase of age. Thus, the mouse model prepared by knocking out the CCL4 gene can be used to simulate and predict the benefits of administering various CCL4 antagonists to animals.

### Example 2: Determination of Wound Healing and Evaluation of Changes in the Wound Area

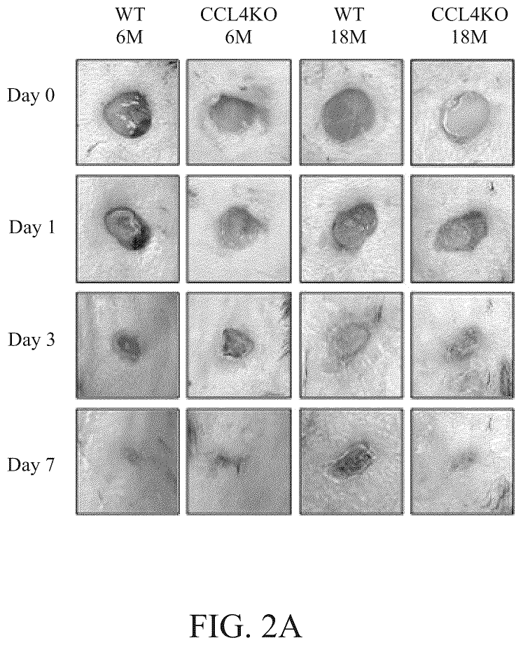

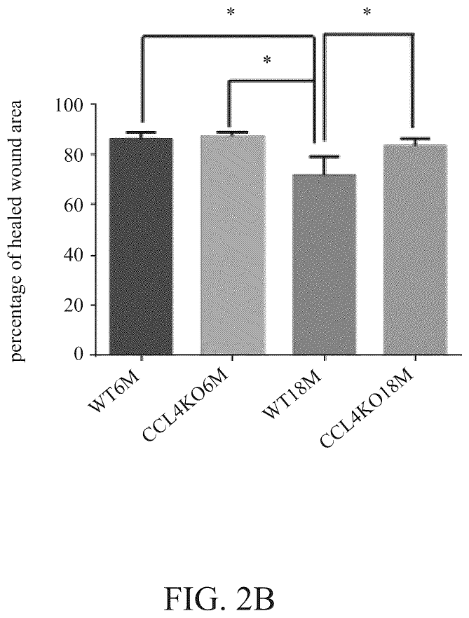

Mice from the WT6M group, CCL4KO6M group, WT18M group, and CCL4KO18M group were full-thickness resected by a biopsy punch to create circular wounds with a diameter of 3 mm, and the wounds did not damage the muscles. Conditions of the wounds were recorded with a digital camera (Nikon, Tokyo, Japan) on days 0, 1, 3 and 7 after the wound were created, and the healed wound area % was calculated on day 7. The results are shown in FIG. 2A and FIG. 2B (n=6, * represents p<0.05).

As shown in FIG. 2A and FIG. 2B, the wound healing rate of aging wild-type mice (WT18M group) was significantly slower than that of other groups. On day 7, the wounds of young mice (WT6M group CCL4KO6M group) have healed by more than 80%, but wounds of WT18M group healed about 70% only. By inhibiting CCL4, the wound healing rate of CCL4KO18M group could be restored to that of young mice. The foregoing results show that aging skin would slow down wound healing rate, while wound healing can be effectively promoted by inhibiting CCL4, and thus achieving the effect of treating or preventing skin aging in an individual.

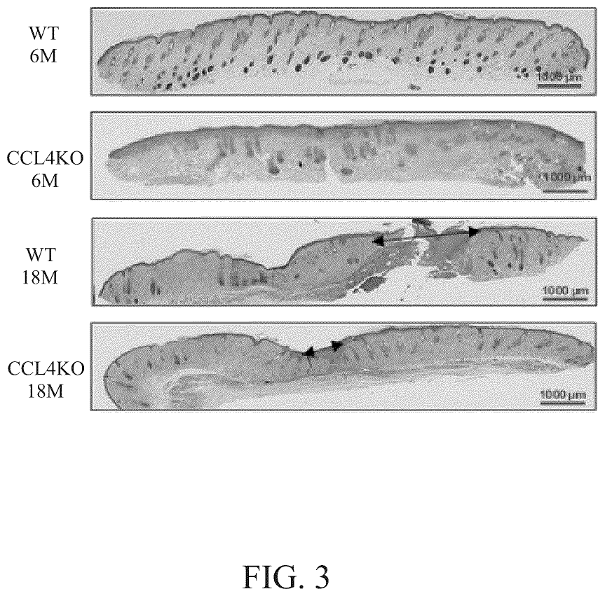

Afterwards, cross-section sections were obtained from the wound areas of mice in each group; fixed with 10% formaldehyde; embedded in paraffin; and stained with hematoxylin/eosin (H&E) on glass slides. The foregoing staining was used to evaluate the changes of the wound type in the wound area, and the results are shown in FIG. 3 (arrows represent the locations and sizes of the wounds).

As shown in FIG. 3, on day 7, almost no obvious wounds were visible on the sections of young mice (WT6M group and CCL4KO6M group), while the wound size of aging wild-type mice (WT18M group) was still quite large. By inhibiting CCL4, the wound size of CCL4KO18M group was significantly smaller than that of WT18M group. The foregoing results once again show that inhibiting CCL4 can promote wound healing effectively and achieve the effect of treating or preventing skin aging in an individual.

### Example 3: Microvessel Density, Cell Proliferation and Collagen Accumulation in Wound Area

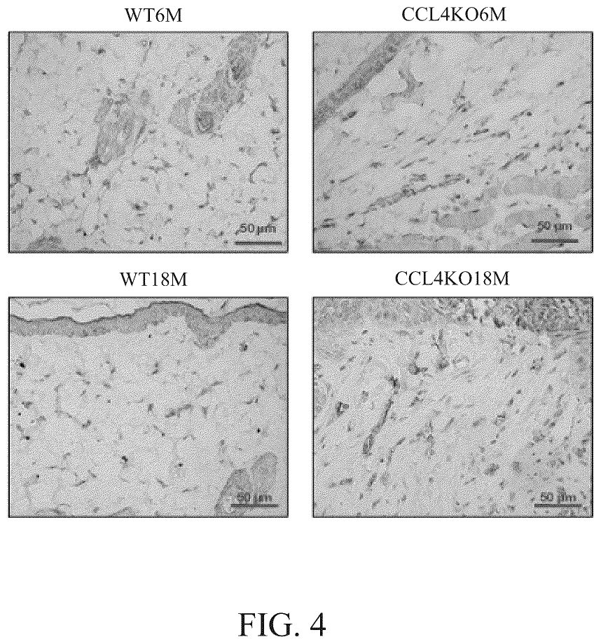

The sections obtained in Example 2 were deparaffinized and cultured with rabbit anti-mouse CD31 (microvascular indicator) polyclonal antibody (Abcam, 124432; Waltham, MA, USA) and rabbit anti-mouse Ki67 (proliferation indicator) polyclonal antibody (Novus, NB500-170; Minneapolis, MN, USA). Antibody distribution was visualized by avidin-biotin-complex technology and Vector Red chromogenic material, and followed by hematoxylin counterstaining. The sections were dried overnight and stained with H&E and Masson's trichrome staining for tissue analysis. The results are shown in FIGS. 4 to 6.

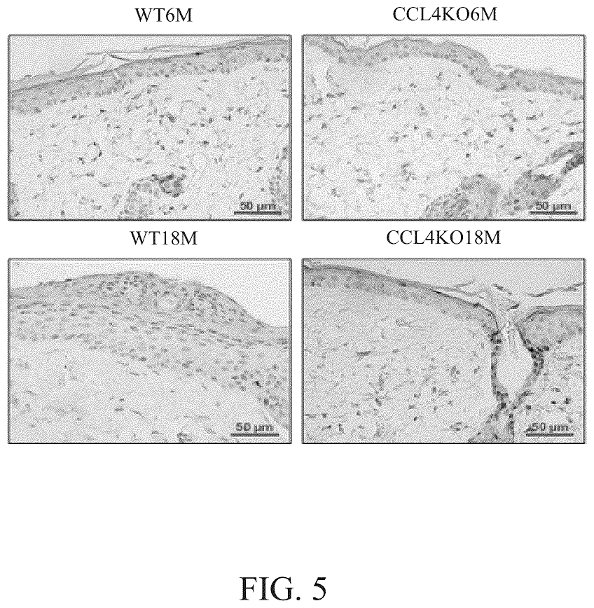

As shown in FIGS. 4 and 5, comparing to aging wild-type mice (WT18M group), aging mice with CCL4 inhibition (CCL4KO18M group) showed larger positive areas in either CD31 (FIG. 4) or Ki67 (FIG. 5). The foregoing results show that inhibiting CCL4 can promote proliferation of microvessel and cells effectively, thereby promoting wound healing and achieving the effect of treating or preventing skin aging in an individual.

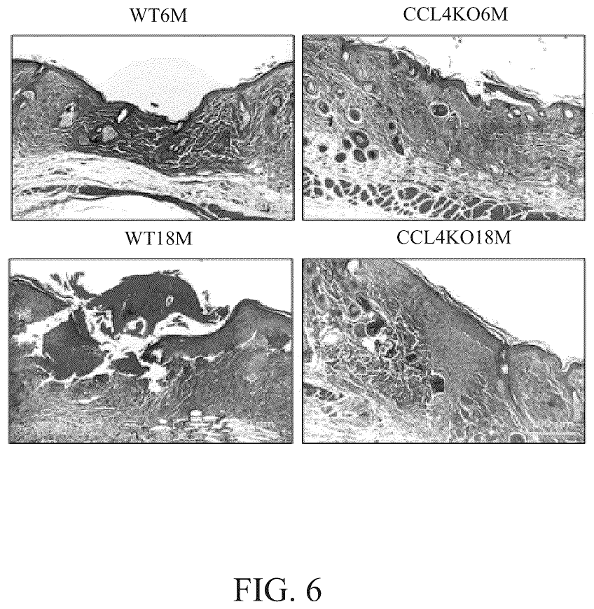

As shown in FIG. 6, comparing to aging wild-type mice (WT18M group), aging mice with CCL4 inhibition (CCL4KO18M group) showed higher content of collagen in the wound area. The foregoing results show that inhibiting CCL4 can increase content of collagen effectively and achieve the effect of treating or preventing skin aging.

### Example 4: Determination of Angiogenesis Factors, Aging Factors, and Inflammatory Factors

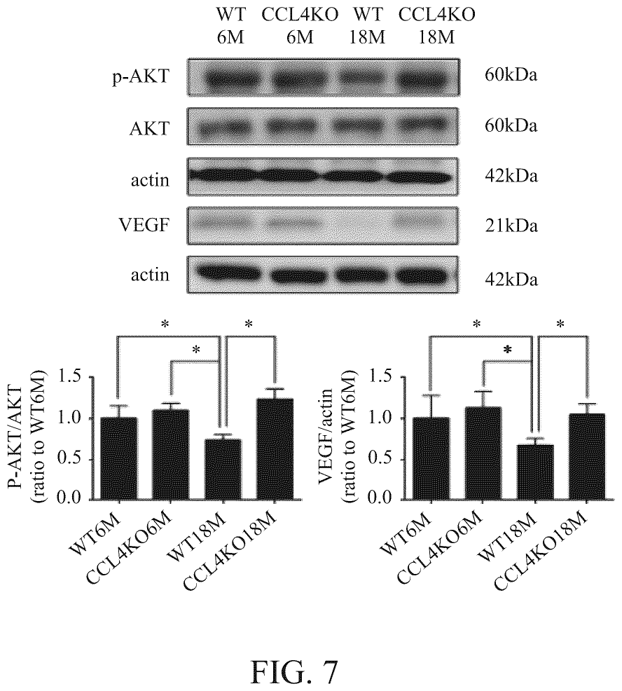

Total cell or tissue lysates were extracted using lysis buffer and proteins were separated on an 8-12% (v/v) SDS-PAGE gel. Following electrophoresis (Bio-Rad Laboratories, Hercules, CA, USA), proteins were transferred to nitrocellulose membrane (Millipore, Darmstadt, Germany), and immunostained with antibodies overnight at 4° C., antibodies include anti-phospho-Akt (BD Biosciences, 550747; NJ, USA), anti-AKT (BD Biosciences, 610868; NJ, USA), anti-VEGF (Santa Cruz Biotechnology, sc-152; Dallas, TX, USA), anti-IL-6 (Cell Signaling, 12153S; Boston, MA, USA), anti-TNF-α (Cell Signaling, 3707S; Boston, MA, USA), anti-SIRT1 (Cell Signaling, 8469S; Boston, MA, USA), anti-p53 (Cell Signaling, 2524S; Boston, MA, USA), and anti-actin (Merck, MAB1501, Darmstadt, Germany). The content of the foregoing proteins was determined by western blot method. P-AKT was normalized by the content of AKT, and other factors were normalized by the content of actin. The results are shown in FIGS. 7 to 9 (n=3, * represents p<0.05, and ** represents p<0.01).

As shown in FIG. 7, comparing to aging wild-type mice (WT18M group), aging mice with CCL4 inhibition (CCL4KO18M group) had higher angiogenic factors (p-AKT (relative to AKT), and VEGF) related protein content. The amount was even comparable to that of young mice (WT6M group and CCL4KO6M group). The foregoing results show that inhibiting CCL4 can promote angiogenesis effectively and achieve the effect of treating or preventing skin aging.

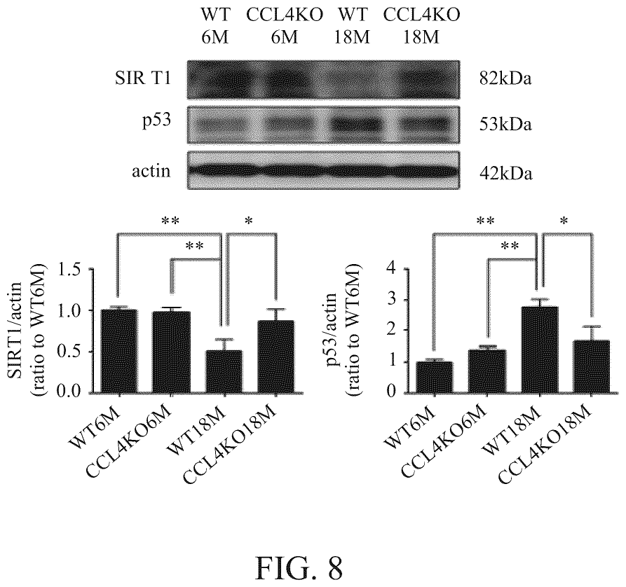

As shown in FIG. 8, comparing to aging wild-type mice (WT18M group), aging mice with CCL4 inhibition (CCL4KO18M group) had higher SIRT1 protein content (the protein was translated by longevity-related genes called SIRT1) and lower p53 protein content (aging factor). The amounts can be restored to be comparable to that of young mice (WT6M group and CCL4KO6M group). The foregoing results show that inhibiting CCL4 can increase longevity factors effectively and reduce the expression of aging factors, thereby achieving the effect of treating or preventing skin aging.

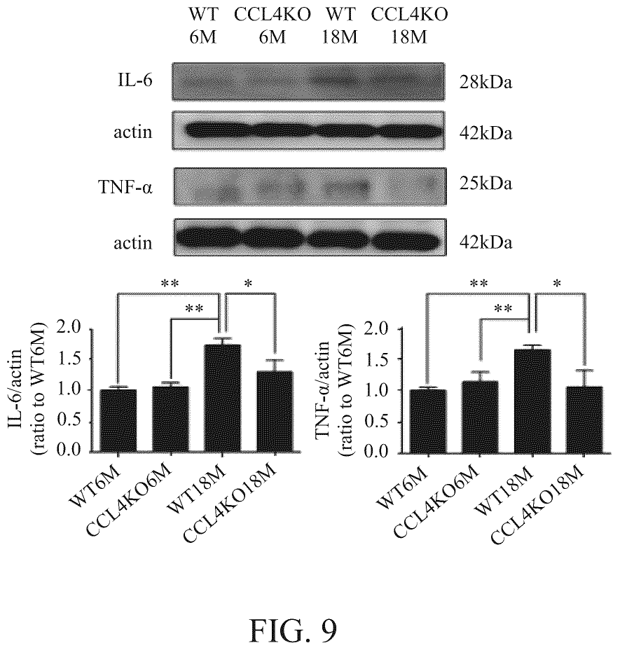

As shown in FIG. 9, comparing to aging wild-type mice (WT18M group), aging mice with CCL4 inhibition (CCL4KO18M group) had lower inflammatory factors (IL-6, and TNF-α) related protein content. The foregoing results show that inhibiting CCL4 can reduce the expression of inflammatory factors effectively and achieve the effect of treating or preventing skin inflammation.

### Example 5: Using Neutralizing Antibody to Verify the Effect of CCL4 Antagonist in Treating or Preventing Skin Aging or Skin Inflammation and Increasing the Content of Collagen

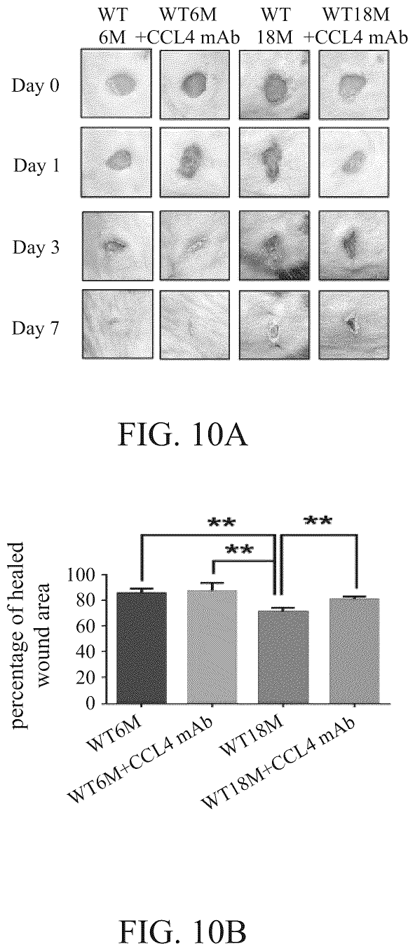

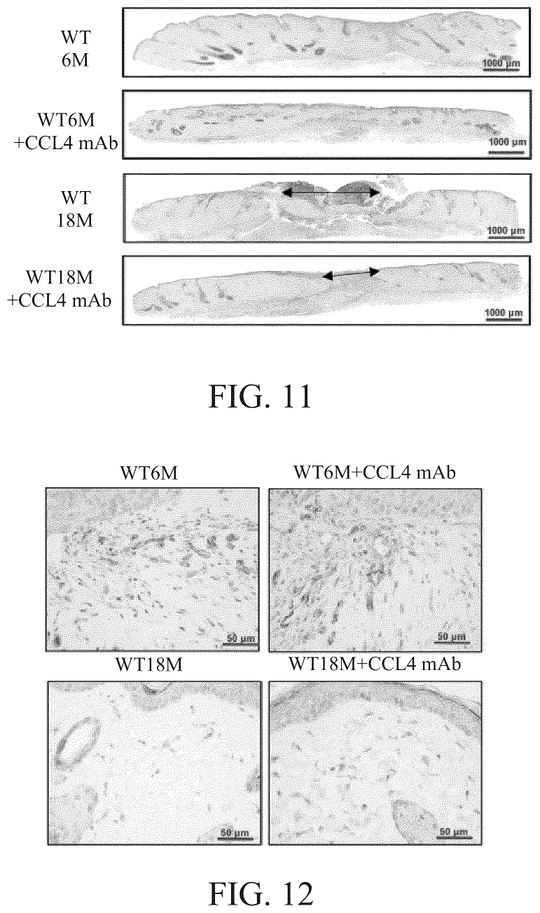

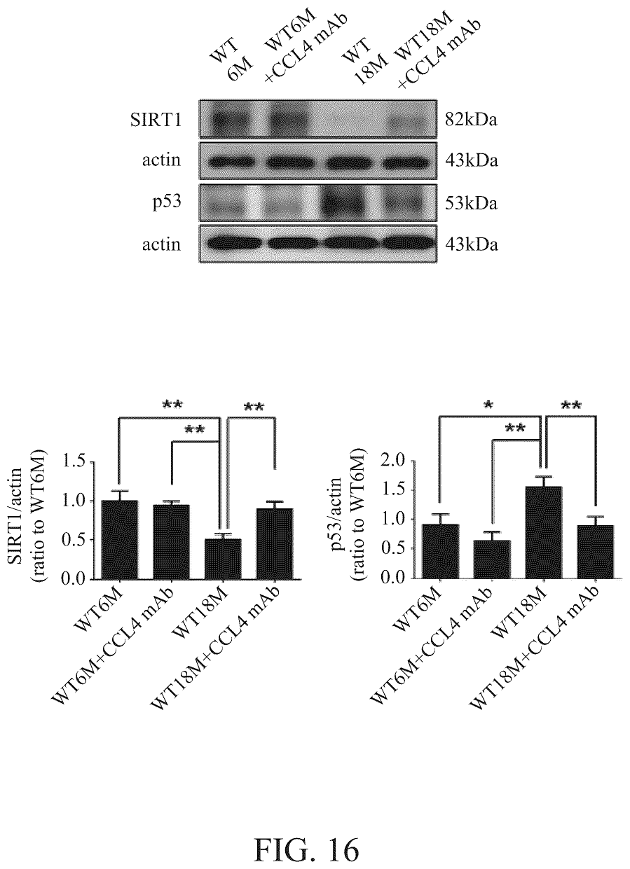

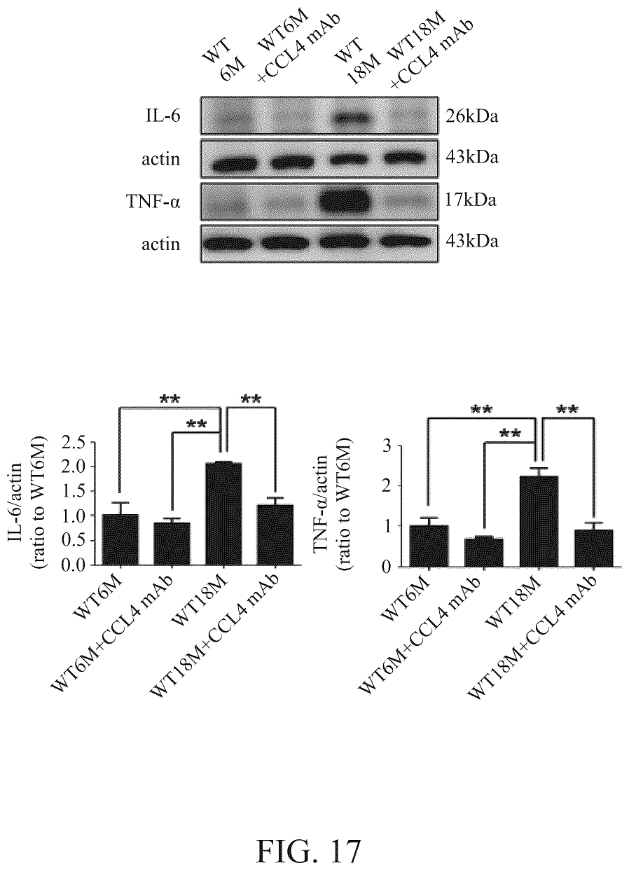

Experimentation of Examples 2 to 4 were repeated in the mice of WT6M group, WT6M+CCL4 mAb group, WT18M group, and WT18M+CCL4 mAb group established in the above example “Preparation of animal models (2). Results are shown respectively in FIG. 10A to FIG. 17. As shown in FIG. 10A and FIG. 10B (n=6), in the wound healing determination, the wound healing rate of aging wild-type mice (WT18M group) was significantly slower than that of other groups. By administering CCL4 neutralizing antibody, the wound healing rate in the WT18M+CCL4 mAb group improved significantly. In the changes of the wound type in the wound area shown in FIG. 11, on day 7, almost no obvious wound was visible on the H&E stained sections of young mice (WT6M group and WT6M+CCL4 mAb group), while the wound size of aging wild-type mice (WT18M group) was still quite large. After administering CCL4 neutralizing antibody, the wound size of WT18M+CCL4 mAb group was significantly smaller than that of WT18M group. The foregoing results once again showed that inhibiting CCL4 can promote wound healing effectively and achieve the effect of treating or preventing skin aging in an individual.

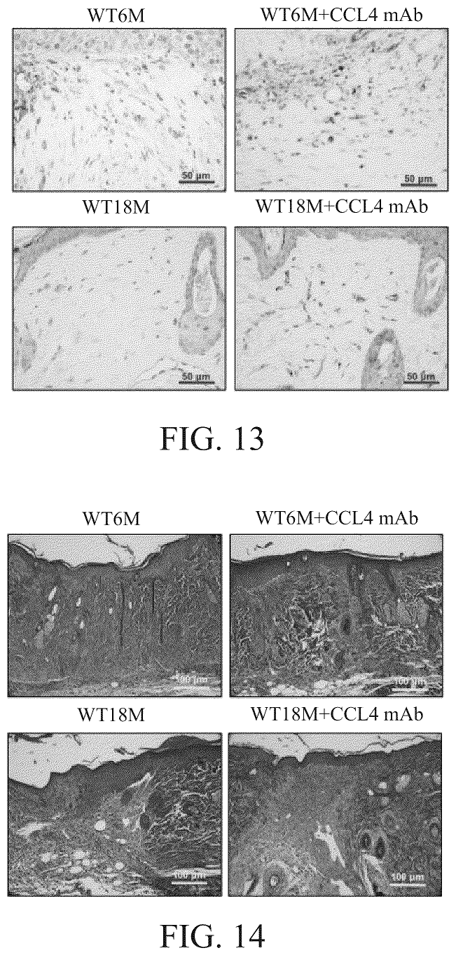

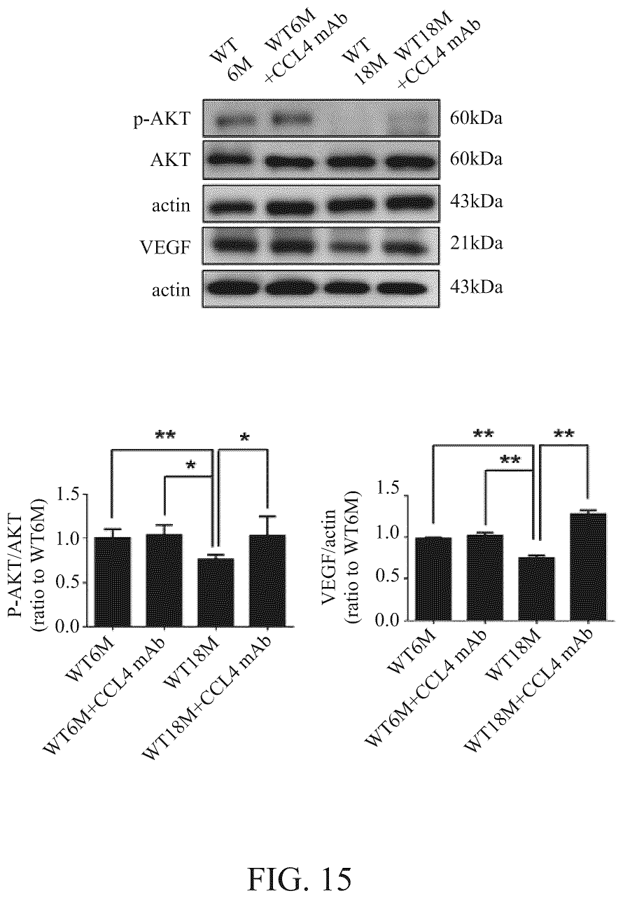

As for microvessel density, cell proliferation and collagen accumulation in wound area, FIGS. 12 to 14 show that comparing to aging wild-type mice (WT18M group), aging mice administered with CCL4 neutralizing antibody (WT18M+CCL4 mAb group) showed larger positive area in either CD31, an indicator of microvessels proliferation (FIG. 12), or Ki67, an indicator of cell proliferation (FIG. 13). Also, as shown in FIG. 14, comparing to aging wild-type mice (WT18M group), aging mice administered with CCL4 neutralizing antibody (WT18M+CCL4 mAb group) showed higher content of collagen in the wound area. The foregoing results showed that inhibiting CCL4 can promote microvessel and cell proliferation, increase content of collagen effectively and achieve the effect of treating or preventing skin aging.

FIGS. 15 to 17 (n=3) show respectively that comparing to aging wild-type mice (WT18M group), aging mice administered with CCL4 neutralizing antibody (WT18M+CCL4 mAb group) had higher angiogenic factors (p-AKT (compared to AKT), and VEGF) related protein content, higher longevity factors (SIRT1) related protein content, lower aging factor (p53) related protein content, and lower inflammatory factors (IL-6, and TNF-α) related protein content. The amounts can even be restored to be comparable to those of young mice (WT6M group and WT6M+CCL4 mAb group).

The above experimental results have been verified repeatedly by different means such as gene knock out and administration of CCL4 antagonist to illustrate that inhibiting CCL4 can be effective for promoting angiogenesis, promoting wound healing, increasing the content of collagen, reducing the expression of inflammatory factors, and/or reducing the expression of aging factors, thereby achieving the effect of treating or preventing skin aging or skin inflammation. Therefore, those with ordinary knowledge in the art should understand that administering any CCL4 antagonist (any antibody, ribonucleic acid, small molecule protein, compound, etc. that can inhibit CCL4 activity) to an individual can achieve the effect of inhibiting CCL4, thereby treating or prevent skin aging or skin inflammation, and increasing content of collagen.

The above descriptions of specific embodiments are merely illustrative of the content of the present disclosure. However, the scope of the present disclosure is not limited to the embodiments disclosed above. It is understood that the specification and embodiments are intended to be exemplary, and therefore the scope of the claims of the present patent application should be given the broadest explanation so as to cover all modifications and changes in accordance with the principles of the present disclosure.

What is claimed is:

1. A method for treating or preventing skin aging or skin inflammation in an individual in need thereof, which includes administering an effective amount of a pharmaceutical composition to the individual, and the pharmaceutical composition includes a CCL4 antagonist and/or a pharmaceutically acceptable salt thereof, and a pharmaceutically acceptable carrier thereof.

2. The method of claim 1, wherein the CCL4 antagonist is at least one selected from the group consisting of a CCL4 neutralizing antibody, a CCL4 RNAi agent, a CCL4 small molecule antagonist, a CCR1 antagonist, a CCR2 antagonist and a CCR5 antagonist.

3. The method of claim 1, wherein the skin aging includes skin laxity, dry skin, rough skin or slow wound healing.

4. The method of claim 1, wherein the pharmaceutical composition promotes angiogenesis, promotes wound healing, increases a content of collagen, reduces expression of an inflammatory factor, and/or reduces expression of an aging factor.

5. The method of claim 1, wherein the pharmaceutical composition is in a form for oral administration, intravenous injection, hypodermic injection, intradermic injection, intramuscular injection, intraperitoneal injection, or transdermal administration.

6. The method of claim 1, wherein the CCL4 antagonist and/or the pharmaceutically acceptable salt thereof is present in the pharmaceutical composition in an amount of 0.01 to 80 wt % based on a total weight of the pharmaceutical composition.

7. The method of claim 1, wherein the CCL4 antagonist and/or the pharmaceutically acceptable salt thereof is administered in an amount of 0.01 mg/kg to 100 mg/kg.

8. The method of claim 1, wherein the pharmaceutical composition is administered 1 to 25 times per week.

9. A method for increasing a content of collagen in an individual in need thereof, which includes administering an effective amount of a cosmetic composition to the individual, and the cosmetic composition includes a CCL4 antagonist and/or a cosmetically acceptable salt thereof and a cosmetically acceptable carrier thereof.

10. The method of claim 9, wherein the CCL4 antagonist is at least one selected from the group consisting of a CCL4 neutralizing antibody, a CCL4 RNAi agent, a CCL4 small molecule antagonist, a CCR1 antagonist, a CCR2 antagonist, and a CCR5 antagonist.

11. The method of claim 9, wherein the CCL4 antagonist and/or the cosmetically acceptable salt thereof is present in the cosmetic composition in an amount of 0.01 to 15 wt % based on the total weight of the cosmetic composition.


SEQUENCE LISTING (2 sequences)

SEQ ID NO: 1 - DNA, 22 bp, synthetic construct
  source 1..22: mol_type = other DNA
  misc_feature 1..22: note = PCR_primers_fwd_seq
tctccctcctttctcttccgtg

SEQ ID NO: 2 - DNA, 23 bp, synthetic construct
  source 1..23: mol_type = other DNA
  misc_feature 1..23: note = PCR_primers_rev_seq
tctactcccaatgatggctgacc